(19) United States
(12) Patent Application Publication
(10) Pub. No.: US 2011/0118309 A1
(43) Pub. Date: May 19, 2011

(54) USE OF HDAC INHIBITORS FOR THE TREATMENT OF HODGKIN'S DISEASE

(76) Inventor: Peter Wisdom Atadja, Acton, MA (US)

(21) Appl. No.: 13/054,528

(22) PCT Filed: Jul. 16, 2009
(86) PCT No.: PCT/US09/50768

§ 371 Date: Jan. 17, 2011

Related U.S. Application Data
(60) Provisional application No. 61/081,805, filed on Jul. 18, 2008.

(51) Int. Cl. A61K 31/47; A61K 31/404; A61P 35/00

(52) U.S. Cl. 514/312; 514/419

(57) ABSTRACT

The present invention relates to the use of an HDAC inhibitor, especially an HDAC inhibitor of formula (I):

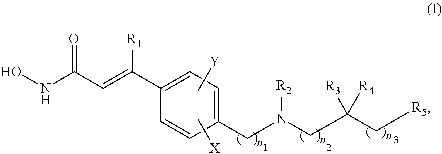

wherein the radicals and symbols have the meanings as defined in the specification, for the preparation of a medicament for the treatment of Hodgkins disease.

The present invention relates to the use of an HDAC inhibitor for the preparation of a medicament for the treatment of Hodgkins; a method of treating a warm-blooded animal, especially a human, having Hodgkins disease, comprising administering to said animal a therapeutically effective amount of an HDAC inhibitor, especially a compound of formula (I) as defined herein; and to a pharmaceutical composition and a commercial package comprising said combination.

The term “Hodgkins disease”, also known as Hodgkin's lymphoma, as used herein is a type of lymphoma first described by Thomas Hodgkin in 1832. Hodgkin's lymphoma is characterized clinically by the orderly spread of disease from one lymph node group to another and by the development of systemic symptoms with advanced disease.

The compounds of formula (I), as defined herein, are histone deacetylase inhibitors (HDAC inhibitors). Reversible acetylation of histones is a major regulator of gene expression that acts by altering accessibility of transcription factors to DNA. In normal cells, histone deacetylase (HDA) and histone acetyltrasferase together control the level of acetylation of histones to maintain a balance. Inhibition of HDA results in the accumulation of hyperacetylated histones, which results in a variety of cellular responses.

Surprisingly, it was now found that HDAC inhibitors, especially the compounds of formula (I), as defined herein, are useful in treatment of Hodgkins disease.

Hence, the invention relates to the use of an HDAC inhibitor for the preparation of a medicament for the treatment of Hodgkins disease.

### HDAC Inhibitor Compounds

HDAC inhibitor compounds of particular interest for use in the inventive combination are hydroxamate compounds described by the formula (I):

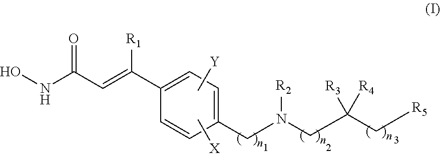

wherein
R₁ is H; halo; or a straight-chain C₁-C₆alkyl, especially methyl, ethyl or n-propyl, which methyl, ethyl and n-propyl substituents are unsubstituted or substituted by one or more substituents described below for alkyl substituents;
R₂ is selected from H; C₁-C₁₀alkyl, preferably C₁-C₆alkyl, e.g., methyl, ethyl or —CH₂CH₂—OH; C₄-C₉cycloalkyl; C₄-C₉heterocycloalkyl; C₄-C₉heterocycloalkylalkyl; cycloalkylalkyl, e.g., cyclopropylmethyl; aryl; heteroaryl; arylalkyl, e.g., benzyl; heteroarylalkyl, e.g., Pyridylmethyl; —(CH₂)ₙC(O)R₆; —(CH₂)ₙOC(O)R₆; amino acyl; HON—C(O)—CH═C(R₁)-aryl-alkyl-; and —(CH₂)ₙR₇;
R₃ and R₄ are the same or different and, independently, H; C₁-C₆alkyl; acyl; or acylamino; or
R₃ and R₄, together with the carbon to which they are bound, represent C═O, C═S or C═NR₈; or
R₂, together with the nitrogen to which it is bound, and R₃, together with the carbon to which it is bound, can form a C₄-C₉heterocycloalkyl; a heteroaryl; a polyheteroaryl; a non-aromatic polyheterocycle; or a mixed aryl and non-aryl polyheterocycle ring;
R₅ is selected from H; C₁-C₆alkyl; C₄-C₉cycloalkyl; C₄-C₉heterocycloalkyl; acyl; aryl; heteroaryl; arylalkyl, e.g., benzyl; heteroarylalkyl, e.g., pyridylmethyl; aromatic polycycles; non-aromatic polycycles; mixed aryl and non-aryl polycycles; polyheteroanyl; non-aromatic polyheterocycles; and mixed aryl and non-aryl polyheterocycles;
n, n₁, n₂ and n₃ are the same or different and independently selected from 0-6, when n₁ is 1-6, each carbon atom can be optionally and independently substituted with R₃ and/or R₄;
X and Y are the same or different and independently selected from H; halo; C₁-C₄alkyl, such as CH₃ and CF; NO₂; C(O)R₁; OR₉; SR₉; CN; and NR₁₀R₁₁;
R₆ is selected from H; C₁-C₆alkyl; C₄-C₉cycloalkyl; C₄-C₉heterocycloalkyl; cycloalkylalkyl, e.g., cyclopropylmethyl; aryl; heteroaryl; arylalkyl, e.g., benzyl and 2-phenylethenyl; heteroarylalkyl, e.g., pyridylmethyl; OR₁₂; and NR₁₃R₁₄;
R₇ is selected from OR₁₅; SR₁₅; S(O)R₁₆; SO₂R₁₇; NR₁₃R₁₄; and NR₁₂SO₂R₆;
R₈ is selected from H; OR; NR₁₃R₁₄; C₁-C₆alkyl; C₄-C₉cycloalkyl; C₄-C₉heterocycloalkyl; aryl; heteroaryl; arylalkyl, e.g., benzyl; and heteroarylalkyl, e.g., pyridylmethyl;
R₉ is selected from C₁-C₄alkyl, e.g., CH₃ and CF; C(O)-alkyl, e.g., C(O)CH₃; and C(O)CF₃;
R₁₀ and R₁₁ are the same or different and independently selected from H; C₁-C₄alkyl; and —C(O)-alkyl;
R₁₂ is selected from H; C₁-C₆alkyl; C₄-C₉cycloalkyl; C₄-C₉heterocycloalkyl; C₄-C₉heterocycloalkylalkyl; aryl; mixed aryl and non-aryl polycycle; heteroaryl; arylalkyl, e.g., benzyl; and heteroarylalkyl, e.g., pyridylmethyl;
R₁₃ and R₁₄ are the same or different and independently selected from H; C₁-C₆alkyl; C₄-C₉cycloalkyl; C₄-C₉heterocycloalkyl; aryl; heteroaryl; arylalkyl, e.g., benzyl; heteroarylalkyl, e.g., pyridylmethyl; amino acyl; or
R₁₃ and R₁₄, together with the nitrogen to which they are bound, are C₄-C₉heterocycloalkyl; heteroaryl; polyheteroaryl; non-aromatic polyheterocycle; or mixed aryl and non-aryl polyheterocycle;
R₁₅ is selected from H; C₁-C₆alkyl; C₄-C₉cycloalkyl; C₄-C₉heterocycloalkyl; aryl; heteroaryl; arylalkyl; heteroarylalkyl; and (CH₂)ₘZR₁₂;
R₁₆ is selected from C₁-C₆alkyl; C₄-C₉cycloalkyl; C₄-C₉heterocycloalkyl; aryl; heteroaryl; polyheteroaryl; arylalkyl; heteroarylalkyl; and (CH₂)ₘZR₁₂;
R₁₇ is selected from C₁-C₆alkyl; C₄-C₉cycloalkyl; C₄-C₉heterocycloalkyl; aryl; aromatic polycycles; heteroaryl; arylalkyl; heteroarylalkyl; polyheteroaryl and NR₁₃R₁₄;
m is an integer selected from 0-6; and
Z is selected from 0; NR₁₃; S; and S(O),
   or a pharmaceutically acceptable salt thereof.

As appropriate, “unsubstituted” means that there is no substituent or that the only substituents are hydrogen.

Halo substituents are selected from fluoro, chloro, bromo and iodo, preferably fluoro or chloro.

Alkyl substituents include straight- and branched-C₁-C₆alkyl, unless otherwise noted. Examples of suitable straight- and branched-C₁-C₆alkyl substituents include methyl, ethyl, n-propyl, 2-propyl, n-butyl, sec-butyl, t-butyl and the like. Unless otherwise noted, the alkyl substituents include both unsubstituted alkyl groups and alkyl groups that are substituted by one or more suitable substituents, including unsaturation, i.e., there are one or more double or triple C—C bonds; acyl; cycloalkyl; halo; oxyalkyl; alkylamino; aminoalkyl; acylamino; and OR₁₅, e.g., alkoxy. Preferred substituents for alkyl groups include halo, hydroxy, alkoxy, oxyalkyl, alkylamino and aminoalkyl.

Cycloalkyl substituents include C₃-C₉cycloalkyl groups, such as cyclopropyl, cyclobutyl, cyclopentyl, cyclohexyl and the like, unless otherwise specified. Unless otherwise noted, cycloalkyl substituents include both unsubstituted cycloalkyl groups and cycloalkyl groups that are substituted by one or more suitable substituents, including C₁-C₆alkyl, halo, hydroxy, aminoalkyl, oxyalkyl, alkylamino and OR₁₅, such as alkoxy. Preferred substituents for cycloalkyl groups include halo, hydroxy, alkoxy, oxyalkyl, alkylamino and aminoalkyl.

The above discussion of alkyl and cycloalkyl substituents also applies to the alkyl portions of other substituents, such as, without limitation, alkoxy, alkyl amines, alkyl ketones, arylalkyl, heteroarylalkyl, alkylsulfonyl and alkyl ester substituents and the like.

Heterocycloalkyl substituents include 3- to 9-membered aliphatic rings, such as 4- to 7-membered aliphatic rings, containing from 1-3 heteroatoms selected from nitrogen, sulfur, oxygen. Examples of suitable heterocycloalkyl substituents include pyrrolidyl, tetrahydrofuryl, tetrahydrothiofuranyl, piperidyl, piperazyl, tetrahydropyranyl, morphilino, 1,3-diazapane, 1,4-diazapane, 1,4-oxazepane and 1,4-oxathiapane. Unless otherwise noted, the rings are unsubstituted or substituted on the carbon atoms by one or more suitable substituents, including C₁-C₆alkyl; C₄-C₉cycloalkyl; aryl; heteroaryl; arylalkyl, e.g., benzyl; heteroarylalkyl, e.g., pyridylmethyl; halo; amino; alkyl amino and OR₁₅, e.g., alkoxy. Unless otherwise noted, nitrogen heteroatoms are unsubstituted or substituted by H, C₁-C₄alkyl; arylalkyl, e.g., benzyl; heteroarylalkyl, e.g., pyridylmethyl; acyl; aminoacyl; alkylsulfonyl; and arylsulfonyl.

Cycloalkylalkyl substituents include compounds of the formula —(CH₂)ₙ₅-cycloalkyl, wherein n5 is a number from 1-6. Suitable alkylcycloalkyl substituents include cyclopentylmethyl, cyclopentylethyl, cyclohexylmethyl and the like. Such substituents are unsubstituted or substituted in the alkyl portion or in the cycloalkyl portion by a suitable substituent, including those listed above for alkyl and cycloalkyl.

Aryl substituents include unsubstituted phenyl and phenyl substituted by one or more suitable substituents including C₁-C₆alkyl; cycloalkylalkyl, e.g., cyclopropylmethyl; O(CO)alkyl; oxyalkyl; halo; nitro; amino; alkylamino; aminoalkyl; alkyl ketones; nitrite; carboxyalkyl; alkylsulfonyl; aminosulfonyl; arylsulfonyl and OR₁₅, such as alkoxy. Preferred substituents include including C₁-C₆alkyl; cycloalkyl, e.g., cyclopropylmethyl; alkoxy; oxyalkyl; halo; nitro; amino; alkylamino; aminoalkyl; alkyl ketones; nitrite; carboxyalkyl; alkylsulfonyl; arylsulfonyl and aminosulfonyl. Examples of suitable aryl groups include C₁-C₄alkylphenyl, C₁-C₄alkoxyphenyl, trifluoromethylphenyl, methoxyphenyl, hydroxyethylphenyl, dimethylaminophenyl, aminopropylphenyl, carbethoxyphenyl, methanesulfonylphenyl and totylsulfonylphenyl.

Aromatic polycycles include naphthyl, and naphthyl substituted by one or more suitable substituents including C₁-C₆alkyl, alkylcycloalkyl, e.g., cyclopropylmethyl; oxyalkyl; halo; nitro; amino; alkylamino; aminoalkyl; alkyl ketones; nitrite; carboxyalkyl; alkylsulfonyl; arylsulfonyl; aminosulfonyl and OR₁₅, such as alkoxy.

Heteroaryl substituents include compounds with a 5- to 7-membered aromatic ring containing one or more heteroatoms, e.g., from 1-4 heteroatoms, selected from N, O and S. Typical heteroaryl substituents include furyl, thienyl, pyrrole, pyrazole, triazole, thiazole, oxazole, pyridine, pyrimidine, isoxazolyl, pyrazine and the like. Unless otherwise noted, heteroaryl substituents are unsubstituted or substituted on a carbon atom by one or more suitable substituents, including alkyl, the alkyl substituents identified above, and another heteroaryl substituent. Nitrogen atoms are unsubstituted or substituted, e.g., by R₁₃; especially useful N substituents include H, acyl, aminoacyl and sulfonyl.

Arylalkyl substituents include groups of the formula —(CH₂)ₙ₅-aryl, —(CH₂)ₙ₅₋₁—(CH-aryl)-(CH₂)ₙ₅-aryl or —(CH₂)ₙ₅₋₁CH(aryl)(aryl), wherein aryl and n5 are defined above. Such arylalkyl substituents include benzyl, 2-phenylethyl, 1-phenylethyl, tolyl-3-propyl, 2-phenylpropyl, diphenylmethyl, 2-diphenylethyl, 5,5-dimethyl-3-phenylpentyl and the like. Arylalkyl substituents are unsubstituted or substituted in the alkyl moiety or the aryl moiety or both as described above for alkyl and aryl substituents.

Heteroarylalkyl substituents include groups of the formula —(CH₂)ₙ₅-heteroaryl, wherein heteroaryl and n5 are defined above and the bridging group is linked to a carbon or a nitrogen of the heteroaryl portion, such as 2-, 3- or 4-pyridylmethyl, imidazolylmethyl, quinolylethyl and pyrrolylbutyl. Heteroaryl substituents are unsubstituted or substituted as discussed above for heteroaryl and alkyl substituents.

Amino acyl substituents include groups of the formula —C(O)—(CH₂)ₙ—C(H)(NR₁₃R₁₄)—(CH₂)ₙ—R₅, wherein n, R₁₃, R₁₄ and R₅ are described above. Suitable aminoacyl substituents include natural and non-natural amino acids, such as glycinyl, D-tryptophanyl, L-lysinyl, D- or L-homoserinyl, 4-aminobutryic acyl and ±-3-amin-4-hexenoyl.

Non-aromatic polycycle substituents include bicyclic and tricyclic fused ring systems where each ring can be 4- to 9-membered and each ring can contain zerio, one or more double and/or triple bonds. Suitable examples of non-aromatic polycycles include decalin, octahydroindene, perhydrobenzocycloheptene and perhydrobenzo-Vi-azulene. Such substituents are unsubstituted or substituted as described above for cycloalkyl groups.

Mixed aryl and non-aryl polycycle substituents include bicyclic and tricyclic fused ring systems where each ring can be 4- to 9-membered and at least one ring is aromatic. Suitable examples of mixed aryl and non-aryl polycycles include methylenedioxyphenyl, bis-methylenedioxyphenyl, 1,2,3,4-tetrahydronaphthalene, dibenzosuberane, dihdydroanthracene and 9H-fluorene. Such substituents are unsubstituted or substituted by nitro or as described above for cycloalkyl groups.

Polyheteroaryl substituents include bicyclic and tricyclic fused ring systems where each ring can independently be 5- or 6-membered and contain one or more heteroatom, e.g., 1, 2, 3 or 4 heteroatoms, chosen from O, N or S such that the fused ring system is aromatic. Suitable examples of polyheteroaryl ring systems include quinoline, isoquinoline, pyridopyrazine, pyrrolopyridine, furopyridine, indole, benzofuran, benzothiofuran, benzindole, benzoxazole, pyrroloquinoline and the like. Unless otherwise noted, polyheteroaryl substituents are unsubstituted or substituted on a carbon atom by one or more suitable substituents, including alkyl, the alkyl substituents identified above and a substituent of the formula —O—(CH₂CH═CH(CH₃)(CH₂))₁₋₃H. Nitrogen atoms are unsubstituted or substituted, e.g., by R₁₃, especially useful N substituents include H, C₁-C₄alkyl, acyl, aminoacyl and sulfonyl.

Non-aromatic polyheterocyclic substituents include bicyclic and tricyclic fused ring systems where each ring can be 4- to 9-membered, contain one or more heteroatom, e.g., 1, 2, 3 or 4 heteroatoms, chosen from O, N or S and contain zero or one or more C—C double or triple bonds. Suitable examples of non-aromatic polyheterocycles include hexitol, cis-perhydro-cyclohepta[b]pyridinyl, decahydro-benzo[f][1,4]oxazepinyl, 2,8-dioxabicyclo[3.3.0]octane, hexahydro-thieno[3,2-b]thiophene, perhydropyrrolo[3,2-b]pyrrole, perhydronaphthyridine, perhydro-1H-dicyclopenta[b,e]pyran. Unless otherwise noted, non-aromatic polyheterocyclic substituents are unsubstituted or substituted on a carbon atom by one or more substituents, including alkyl and the alkyl substituents identified above. Nitrogen atoms are unsubstituted or substituted, e.g., by R₁₃, especially useful N substituents include H, C₁-C₄alkyl, acyl, aminoacyl and sulfonyl.

Mixed aryl and non-aryl polyheterocycle substituents include bicyclic and tricyclic fused ring systems where each ring can be 4- to 9-membered, contain one or more heteroatom chosen from O, N or S, and at least one of the rings must be aromatic. Suitable examples of mixed aryl and non-aryl polyheterocycles include 2,3-dihydroindole, 1,2,3,4-tetrahydroquinoline, 5,11-dihydro-10H-dibenz[b,e][1,4]diazepine, 5H-dibenzo[b,e][1,4]diazepine, 1,2-dihydropyrrolo[3,4-b][1,5]benzodiazepine, 1,5-dihydro-pyrido[2,3-b][1,4]diazepin-4-one, 1,2,3,4,6,11-hexahydro-benzo[b]pyrido[2,3-e][1,4]diazepin-5-one. Unless otherwise noted, mixed aryl and non-aryl polyheterocyclic substituents are unsubstituted or substituted on a carbon atom by one or more suitable substituents including —N—OH, ═N—OH, alkyl and the alkyl substituents identified above. Nitrogen atoms are unsubstituted or substituted, e.g., by R₁₃; especially useful N substituents include H, C₁-C₄alkyl, acyl, aminoacyl and sulfonyl.

Amino substituents include primary, secondary and tertiary amines and in salt form, quaternary amines. Examples of amino substituents include mono- and di-alkylamino, mono- and di-aryl amino, mono- and di-arylalkyl amino, aryl-arylalkylamino, alkyl-arylamino, alkyl-arylalkylamino and the like.

Sulfonyl substituents include alkylsulfonyl and arylsulfonyl, e.g., methane sulfonyl, benzene sulfonyl, tosyl and the like.

Acyl substituents include groups of formula —C(O)—W, —OC(O)—W, —C(O)—O—W or —C(O)NR₁₃R₁₄, where W is R₁₆, H or cycloalkylalkyl.

Acylamino substituents include substituents of the formula —N(R₁₂)C(O)—W, —N(R₁₂)C(O)—O—W and —N(R₁₂)C(O)—NHOH and R₁₂ and W are defined above.

The R₂ substituent HON—C(O)—CH═C(R₁)-aryl-alkyl- is a group of the formula

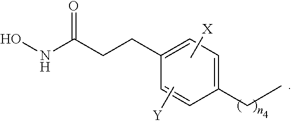

Preferences for each of the substituents include the following:
R₁ is H, halo or a straight-chain C₁-C₄alkyl;
R₂ is selected from H, C₁-C₆alkyl, C₄-C₉cycloalkyl, C₄-C₉heterocycloalkyl, cycloalkylalkyl, aryl, heteroaryl, arylalkyl, heteroarylalkyl, —(CH₂)ₙC(O)R₆, amino acyl and —(CH₂)ₙR₇;
R₃ and R₄ are the same or different and independently selected from H and C₁-C₆alkyl; or
R₃ and R₄, together with the carbon to which they are bound, represent C═O, C═S or C═NR₈;
R₅ is selected from H, C₁-C₆alkyl, C₄-C₉cycloalkyl, C₄-C₉heterocycloalkyl, aryl, heteroaryl, arylalkyl, heteroarylalkyl, a aromatic polycycle, a non-aromatic polycycle, a mixed aryl and non-aryl polycycle, polyheteroaryl, a non-aromatic polyheterocycle, and a mixed aryl and non-aryl polyheterocycle;
n, n₁, n₂ and n₃ are the same or different and independently selected from 0-6, when n₁ is 1-6, each carbon atom is unsubstituted or independently substituted with R₃ and/or R₄;
X and Y are the same or different and independently selected from H, halo, C₁-C₄alkyl, CF₃, NO₂, C(O)R₁, OR₆, SR₉, CN and NR₁₀R₁₁;
R₆ is selected from H, C₁-C₆alkyl, C₄-C₉cycloalkyl, C₄-C₉heterocycloalkyl, alkylcycloalkyl, aryl, heteroaryl, arylalkyl, heteroarylalkyl, OR₁₂ and NR₁₃R₁₄;
R₇ is selected from OR₁₅, SR₁₅, S(O)R₁₆, SO₂R₁₇, NR₁₃R₁₄ and NR₁₂SO₂R₆;
R₈ is selected from H, OR₁₅, NR₁₃R₁₄, C₁-C₆alkyl, C₄-C₉cycloalkyl, C₄-C₉heterocycloalkyl, aryl, heteroaryl, arylalkyl and heteroarylalkyl;
R₉ is selected from C₁-C₄alkyl and C(O)-alkyl;
R₁₀ and R₁₁ are the same or different and independently selected from H, C₁-C₄alkyl and —C(O)-alkyl;
R₁₂ is selected from H, C₁-C₆alkyl, C₄-C₉cycloalkyl, C₄-C₉heterocycloalkyl, aryl, heteroaryl, arylalkyl and heteroarylalkyl;
R₁₃ and R₁₄ are the same or different and independently selected from H, C₁-C₆alkyl, C₄-C₉cycloalkyl, C₄-C₉heterocycloalkyl, aryl, heteroaryl, arylalkyl, heteroarylalkyl and amino acyl;
R₁₅ is selected from H, C₄-C₉cycloalkyl, C₄-C₉heterocycloalkyl, aryl, heteroaryl, arylalkyl, heteroarylalkyl and (CH₂)ₘZR₁₂;
R₁₆ is selected from C₁-C₆alkyl, C₄-C₉cycloalkyl, C₄-C₉heterocycloalkyl, aryl, heteroaryl, arylalkyl, heteroarylalkyl and (CH₂)ₘZR₁₂;
R₁₇ is selected from C₁-C₆alkyl, C₄-C₉cycloalkyl, C₄-C₉heterocycloalkyl, aryl, heteroaryl, arylalkyl, heteroarylalkyl and NR₁₃R₁₄;
m is an integer selected from 0-6; and
Z is selected from O, NR₁₃, S and S(O);
   or a pharmaceutically acceptable salt thereof.

Useful compounds of the formula (I), include those wherein each of R₁, X, Y, R₃ and R₄ is H, including those wherein one of n₂ and n₃ is 0 and the other is 1, especially those wherein R₂ is H or —CH₂—CH₂—OH.

One suitable genus of hydroxamate compounds are those of formula (Ia):

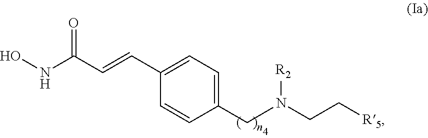

wherein
n₄ is 0-3;
R₂ is selected from H, C₁-C₆cycloalkyl, C₄-C₉heterocycloalkyl, cycloalkylalkyl, aryl, heteroaryl, arylalkyl, heteroarylalkyl, —(CH₂)ₙC(O)R₆, amino acyl and —(CH₂)ₙR₇; and
R₅′ is heteroaryl; heteroarylalkyl, e.g., pyridylmethyl; aromatic polycycles; non-aromatic polycycles; mixed aryl and non-aryl polycycles; polyheteroaryl or mixed aryl; and non-aryl polyheterocycles;
   or a pharmaceutically acceptable salt thereof,

Another suitable genus of hydroxamate compounds are those of formula (Ia):

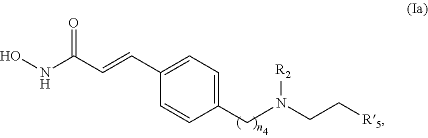

wherein
n₄ is 0-3;
R₂ is selected from H, C₁-C₆cycloalkyl, C₄-C₉heterocycloalkyl, cycloalkylalkyl, aryl, heteroaryl, arylalkyl, heteroarylalkyl, —(CH₂)ₙC(O)R₆, amino acyl and —(CH₂)ₙR₇;
R₅′ is aryl; arylalkyl: aromatic polycycles; non-aromatic polycycles and mixed aryl; and non-aryl polycycles, especially aryl, such as p-fluorophenyl, p-chlorophenyl, p-O—C₁-C₄alkylphenyl, such as p-methoxyphenyl, and p-C₁-C₄alkylphenyl: and arylalkyl, such as benzyl, ortho-, meta- or para-fluorobenzyl, ortho-, meta- or para-chlorobenzyl, ortho-, meta- or para-mono, di- or tri-O—C₁-C₄alkylbenzyl, such as ortho-, meta- or para-methoxybenzyl, m,p-diethoxybenzyl, o,m,p-triimethoxybenzyl and ortho-, meta- or para-mono, di- or tri-C₁-C₄alkylphenyl, such as p-methyl, m,m-diethylphenyl,
   or a pharmaceutically acceptable salt thereof.

Another interesting genus is the compounds of formula (Ib);

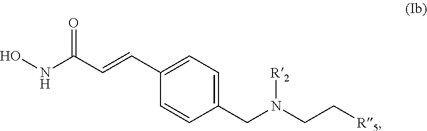

wherein
R₂′ is selected from H; C₁-C₆alkyl; C₄-C₆cycloalkyl; cycloalkylalkyl, e.g., cyclopropylmethyl; (CH₂)₂₋₄OR₂₁, where R₂₁ is H, methyl, ethyl, propyl and i-propyl; and
R₅″ is unsubstituted benzofuran-3-yl or quinolin-3-yl, or substituted 1H-indol-3-yl, such as 5-fluoro-1H-indol-3-yl or 5-methoxy-1H-indol-3-yl, benzofuran-3-yl or quinolin-3-yl;
   or a pharmaceutically acceptable salt thereof.

Another interesting genus of hydroxamate compounds are the compounds of formula (Ic):

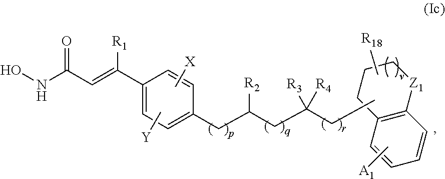

wherein
the ring containing Z₁ is aromatic or non-aromatic, which non-aromatic rings are saturated or unsaturated,
Z₁ is O, S or N—R₂₀;
R₁₈ is H; halo; C₁-C₆alkyl (methyl, ethyl, t-butyl); C₃-C₇cycloalkyl; aryl, e.g., unsubstituted phenyl or phenyl substituted by 4-OCH₃ or 4-CF₃; or heteroaryl, such as 2-furanyl, 2-thiophenyl or 2-, 3- or 4-pyridyl;
R₂₀ is H: C₁-C₆alkyl; C₁-C₆alkyl-C₃-C₉cycloalkyl, e.g., cyclopropylmethyl; aryl; heteroaryl; arylalkyl, e.g., benzyl; heteroarylalkyl, e.g., pyridylmethyl; acyl, e.g., acetyl, propionyl and benzoyl; or sulfonyl, methanesulfonyl, ethanesulfonyl, benzenesulfonyl and toluenesulfonyl;
A₁ is 1, 2 or 3 substituents which are independently H; C₁-C₆alkyl; —OR₁₉; halo; alkylamino; aminoalkyl; halo; or heteroarylalkyl, e.g., pyridylmethyl;
R₁₉ is selected from H; C₁-C₆alkyl; C₄-C₉cycloalkyl; C₄-C₉heterocycloalkyl; aryl; heteroaryl; arylalkyl, e.g., benzyl; heteroarylalkyl, pyridylmethyl and —(CH₂CH═CH(CH₃)(CH₂))₁₋₃H;
R₂ is selected from H, C₁-C₆alkyl, C₄-C₉cycloalkyl, C₄-C₉heterocycloalkyl, cycloalkylalkyl, aryl, heteroaryl, arylalkyl, heteroarylalkyl, —(CH₂)ₙC(O)R₆, amino acyl and —(CH₂)ₙR₇;
v is 0, 1 or 2;
p is 0-3; and
q is 1-5 and r is 0; or
q is 0 and r is 1-5;
   or a pharmaceutically acceptable salt thereof. The other variable substituents are as defined above.

Especially useful compounds of formula (Ic), are those wherein R₂ is H, or —(CH₂)ₚCH₂OH, wherein p is 1-3, especially those wherein R₁ is H; such as those wherein R₁ is H and X and Y are each H, and wherein q is 1-3 and r is 0 or wherein g is 0 and r is 1-3, especially those wherein Z₁ is N—R₂₀. Among these compounds R₂ is preferably H or —CH₂—CH₂—OH and the sum of g and r is preferably 1.

Another interesting genus of hydroxamate compounds are the compounds of formula (Id):

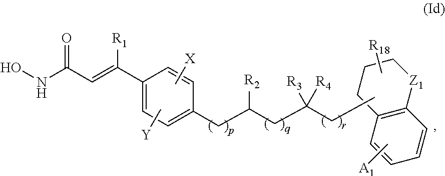

wherein
Z₁ is O, S or N—R₂₀;
R₁₈ is H; halo; C₁-C₆alkyl (methyl, ethyl, t-butyl); C₃-C₇cycloalkyl; aryl, e.g., unsubstituted phenyl or phenyl substituted by 4-OCH₃ or 4-CF₃; heteroaryl;
R₂₀ is H; C₁-C₆alkyl, C₁-C₆alkyl-C₃-C₉cycloalkyl, e.g., cyclopropylmethyl; aryl; heteroaryl; arylalkyl, e.g., benzyl; heteroarylalkyl, e.g., pyridylmethyl; acyl, e.g., acetyl, propionyl and benzoyl; or sulfonyl, e.g., methanesulfonyl, ethanesulfonyl, benzenesulfonyl, toluenesulfonyl);
A₁ is 1, 2 or 3 substituents which are independently H, C₁-C₆alkyl, —OR₁₉ or halo;
R₁₉ is selected from H; C₁-C₆alkyl; C₄-C₉cycloalkyl; C₄-C₉heterocycloalkyl; aryl; heteroaryl; arylalkyl, e.g., benzyl; and heteroarylalkyl, e.g., pyridylmethyl;
p is 0-3; and
q is 1-5 and r is 0; or
q is 0 and r is 1-5;
   or a pharmaceutically acceptable salt thereof. The other variable substituents are as defined above.

Especially useful compounds of formula (Id), are those wherein R₂ is H or —(CH₂)ₚCH₂OH, wherein p is 1-3, especially those wherein R₁ is H; such as those wherein R₁ is H and X and Y are each H, and wherein q is 1-3 and r is 0 or wherein q is 0 and r is 1-3. Among these compounds R₂ is preferably H or —CH₂—CH₂—OH and the sum of q and r is preferably 1.

The present invention further relates to compounds of the formula (Ie):

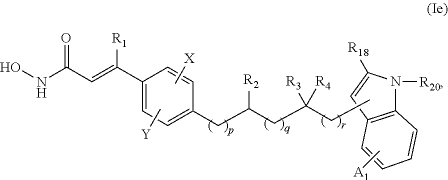

or a pharmaceutically acceptable salt thereof. The variable substituents are as defined above.

Especially useful compounds of formula (Ie), are those wherein R₁₈ is H, fluoro, chloro, bromo, a C₁-C₄alkyl group, a substituted C₁-C₄alkyl group, a C₃-C₇cycloalkyl group, unsubstituted phenyl, phenyl substituted in the pare position, or a heteroaryl e.g., pyridyl, ring.

Another group of useful compounds of formula (Ie), are those wherein R₂ is H or —(CH₂)CH₂OH, wherein p is 1-3, especially those wherein R₁ is H; such as those wherein R₁ is H and X and are each H, and wherein q is 1-3 and r is 0 or wherein q is 0 and r is 1-3. Among these compounds R₂ is preferably H or —CH₂—CH₂—OH and the sum of q and r is preferably 1. Among these compounds p is preferably 1 and R₃ and R₄ are preferably H.

Another group of useful compounds of formula (Ie), are those wherein R₁₈ is H, methyl, ethyl, t-butyl, trifluoromethyl, cyclohexyl, phenyl, 4-methoxyphenyl, 4-trifluoromethylphenyl, 2-furanyl, 2-thiophenyl, or 2-, 3- or 4-pyridyl wherein the 2-furanyl, 2-thiophenyl and 2-, 3- or 4-pyridyl substituents are unsubstituted or substituted as described above for heteroaryl rings; R₂ is H or —(CH₂)ₚCH₂OH, wherein p is 1-3; especially those wherein R₁ is H and X and are each H, and wherein q is 1-3 and r is 0 or wherein q is 0 and r is 1-3. Among these compounds R₂ is preferably H or —CH₂—CH₂—OH and the sum of q and r is preferably 1.

Those compounds of formula (Ie), wherein R₂₀ is H or C₁-C₆alkyl, especially H, are important members of each of the subgenuses of compounds of formula (Ie) described above.

N-hydroxy-3-[4-[[(2-hydroxyethyl)[2-(1H-indol-3-yl)ethyl]-amino]methyl]phenyl]-2E-2-propenamide, N-hydroxy-3-[4-[[[2-(1H-indol-3-yl)ethyl]-amino]methyl]phenyl]-2E-2-propenamide and N-hydroxy-3-[4-[[[2-(2-methyl-1H-indol-3-yl)-ethyl]-amino]methyl]phenyl]-2E-2-propenamide or a pharmaceutically acceptable salt thereof, are important compounds of formula (Ie).

The present invention further relates to the compounds of the formula (If):

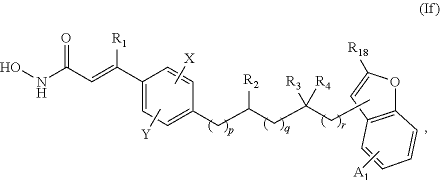

or a pharmaceutically acceptable salt thereof. The variable substituents are as defined above.

Useful compounds of formula (If), are include those wherein R₂ is H or —(CH₂)ₚCH₂OH, wherein p is 1-3, especially those wherein R₁ is H; such as those wherein R₁ is H and X and Y are each H, and wherein q is 1-3 and r is 0 or wherein q is 0 and r is 1-3. Among these compounds R₂ is preferably H or —CH₂—CH₂—OH and the sum of q and r is preferably 1.

N-hydroxy-3-[4-[[[2-(benzofur-3-yl)-ethyl]-amino]methyl]phenyl]-2E-2-propenamide or a pharmaceutically acceptable salt thereof, is an important compound of formula (If).

The compounds described above are often used in the form of a pharmaceutically acceptable salt. Pharmaceutically acceptable salts include, when appropriate, pharmaceutically acceptable base addition salts and acid addition salts, e.g., metal salts, such as alkali and alkaline earth metal salts, ammonium salts, organic amine addition salts and amino acid addition salts and sulfonate salts. Acid addition salts include inorganic acid addition salts, such as hydrochloride, sulfate and phosphate; and organic acid addition salts, such as alkyl sulfonate, arylsulfonate, acetate, maleate, fumarate, tartrate, citrate and lactate. Examples of metal salts are alkali metal salts, such as lithium salt, sodium salt and potassium salt; alkaline earth metal salts, such as magnesium salt and calcium salt, aluminum salt and zinc salt. Examples of ammonium salts are ammonium salt and tetramethylammonium salt. Examples of organic amine addition salts are salts with morpholine and piperidine. Examples of amino acid addition salts are salts with glycine, phenylalanine, glutamic acid and lysine. Sulfonate salts include mesylate, tosylate and benzene sulfonic acid salts.

Additional HDAC compounds within the scope of formula (I), and their synthesis, are disclosed in WO 02/22577 published Mar. 21, 2002 which is incorporated herein by reference in its entirety. Two preferred compounds within the scope of WO 02/22577 are:

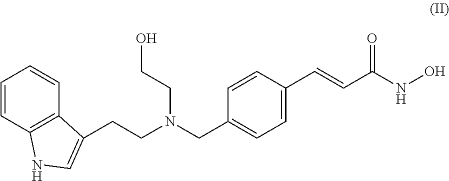

N-hydroxy-3-[4-[(2-hydroxyethyl){2-(1H-indol-3-yl)ethyl]-amino]methyl]phenyl]-2E-2-propenamide, or a pharmaceutically acceptable salt thereof and

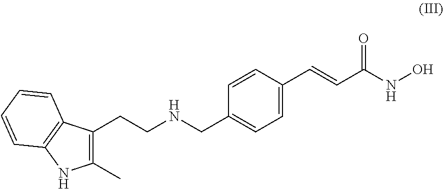

N-hydroxy-3-[4-[[[2-(2-methyl-1H-indol-3-yl)-ethyl]-amino]methyl]phenyl]-2E-2-propenamide, or a pharmaceutically acceptable salt thereof.

Furthermore, the invention relates to a method of treating Hodgkins disease, comprising administering a therapeutically effective amount of an HDAC inhibitor to a warm-blooded animal, in particular a human, in need thereof, preferably a therapeutically effective amount of a compound of formula (I), as defined above, or the salt of such compound having at least one salt-forming group, to a warm-blooded animal, preferably a human, in need thereof.

The term “treatment”, as used herein, comprises the treatment of patients having Hodgkins disease or being in a pre-stage of said disease which effects the delay of progression of the disease in said patients.

The present invention provides a method of treating Hodgkins disease comprising administering a an HDAC inhibitor in an amount which is therapeutically effective against Hodgkins disease to a warm-blooded animal in need thereof.

The person skilled in the pertinent art is fully enabled to select relevant test models to prove the hereinbefore and hereinafter mentioned beneficial effects on Hodgkins disease of a compound inhibiting the HDAC activity. The pharmacological activity of a compound inhibiting the HDAC activity may, e.g., be demonstrated in a suitable clinical study or by means of the Examples described below.

The present invention also provides the use of a compound of formula (I), as defined herein, and the use of a COMBINATION OF THE INVENTION for the preparation of a medicament for the treatment of Hodgkins disease.

Combination partners include antiproliferative compounds. Such antiproliferative compounds include, but are not limited to aromatase inhibitors; antiestrogens; topoisomerase I inhibitors; topoisomerase II inhibitors; microtubule active compounds; alkylating compounds; histone deacetylase inhibitors; compounds which induce cell differentiation processes; cyclooxygenase inhibitors; MMP inhibitors; mTOR inhibitors; antineoplastic antimetabolites; platin compounds; compounds targeting/decreasing a protein or lipid kinase activity and further anti-angiogenic compounds; compounds which target, decrease or inhibit the activity of a protein or lipid phosphatase; gonadorelin agonists; anti-androgens; methionine aminopeptidase inhibitors; bisphosphonates; biological response modifiers; antiproliferative antibodies; heparanase inhibitors; inhibitors of Ras oncogenic isoforms; telomerase inhibitors; proteasome inhibitors; compounds used in the treatment of hematologic malignancies; compounds which target, decrease or inhibit the activity of Flt-3; Hsp90 inhibitors such as 17-AAG (17-allylaminogeldanamycin, NSC330507), 17-DMAG (17-dimethylaminoethylamino-17-demethoxy-geldanamycin, NSC707545), IPI-504, CNF1010, CNF2024, CNF1010 from Conforma Therapeutics; temozolomide (TEMODAL®); kinesin spindle protein inhibitors, such as SB715992 or SB743921 from GlaxoSmithKline, or pentamidine/chlorpromazine from CombinatoRx; MEK inhibitors such as ARRY142886 from Array PioPharma, AZD6244 from AstraZeneca, PD181461 from Pfizer and leucovorin. The term “aromatase inhibitor” as used herein relates to a compound which inhibits the estrogen production, i.e. the conversion of the substrates androstenedione and testosterone to estrone and estradiol, respectively. The term includes, but is not limited to steroids, especially atamestane, exemestane and formestane and, in particular, non-steroids, especially aminoglutethimide, roglethimide, pyridoglutethimide, trilostane, testolactone, ketokonazole, vorozole, fadrozole, anastrozole and letrozole. Exemestane can be administered, e.g., in the form as it is marketed, e.g. under the trademark AROMASIN. Formestane can be administered, e.g., in the form as it is marketed, e.g. under the trademark LENTARON. Fadrozole can be administered, e.g., in the form as it is marketed, e.g. under the trademark AFEMA. Anastrozole can be administered, e.g., in the form as it is marketed, e.g. under the trademark ARIMIDEX. Letrozole can be administered, e.g., in the form as it is marketed, e.g. under the trademark FEMARA or FEMAR. Aminoglutethimide can be administered, e.g., in the form as it is marketed, e.g. under the trademark ORIMETEN. A combination of the invention comprising a chemotherapeutic agent which is an aromatase inhibitor is particularly useful for the treatment of hormone receptor positive tumors, e.g. breast tumors.

The term “antiestrogen” as used herein relates to a compound which antagonizes the effect of estrogens at the estrogen receptor level. The term includes, but is not limited to tamoxifen, fulvestrant, raloxifene and raloxifene hydrochloride. Tamoxifen can be administered, e.g., in the form as it is marketed, e.g. under the trademark NOLVADEX. Raloxifene hydrochloride can be administered, e.g., in the form as it is marketed, e.g. under the trademark EVISTA. Fulvestrant can be formulated as disclosed in U.S. Pat. No. 4,659,516 or it can be administered, e.g., in the form as it is marketed, e.g. under the trademark FASLODEX. A combination of the invention comprising a chemotherapeutic agent which is an antiestrogen is particularly useful for the treatment of estrogen receptor positive tumors, e.g. breast tumors.

The term “anti-androgen” as used herein relates to any substance which is capable of inhibiting the biological effects of androgenic hormones and includes, but is not limited to, bicalutamide (CASODEX), which can be formulated, e.g. as disclosed in U.S. Pat. No. 4,636,505. The term “gonadorelin agonist” as used herein includes, but is not limited to abarelix, goserelin and goserelin acetate. Goserelin is disclosed in U.S. Pat. No. 4,100,274 and can be administered, e.g., in the form as it is marketed, e.g. under the trademark ZOLADEX. Abarelix can be formulated, e.g. as disclosed in U.S. Pat. No. 5,843,901.

The term “topoisomerase I inhibitor” as used herein includes, but is not limited to topotecan, gimatecan, irinotecan, camptothecian and its analogues, 9-nitrocamptothecin and the macromolecular camptothecin conjugate PNU-166148 (compound A1 in WO99/17804). Irinotecan can be administered, e.g. in the form as it is marketed, e.g. under the trademark CAMPTOSAR. Topotecan can be administered, e.g., in the form as it is marketed, e.g. under the trademark HYCAMTIN.

The term “topoisomerase II inhibitor” as used herein includes, but is not limited to the anthracyclines such as doxorubicin (including liposomal formulation, e.g. CAELYX), daunorubicin, epirubicin, idarubicin and nemorubicin, the anthraquinones mitoxantrone and losoxantrone, and the podophillotoxines etoposide and teniposide. Etoposide can be administered, e.g. in the form as it is marketed, e.g. under the trademark ETOPOPHOS. Teniposide can be administered, e.g. in the form as it is marketed, e.g. under the trademark VM 26-BRISTOL. Doxorubicin can be administered, e.g. in the form as it is marketed, e.g. under the trademark ADRIBLASTIN or ADRIAMYCIN. Epirubicin can be administered, e.g. in the form as it is marketed, e.g. under the trademark FARMORUBICIN. Idarubicin can be administered, e.g. in the form as it is marketed, e.g. under the trademark ZAVEDOS. Mitoxantrone can be administered, e.g. in the form as it is marketed, e.g. under the trademark NOVANTRON. The term “microtubule active agent” relates to microtubule stabilizing, microtubule destabilizing compounds and microtublin polymerization inhibitors including, but not limited to taxanes, e.g. paclitaxel and docetaxel, vinca alkaloids, e.g., vinblastine, especially vinblastine sulfate, vincristine especially vincristine sulfate, and vinorelbine, discodermolides, cochicine and epothilones and derivatives thereof, e.g. epothilone B or D or derivatives thereof. Paclitaxel may be administered e.g. in the form as it is marketed, e.g. TAXOL. Docetaxel can be administered, e.g., in the form as it is marketed, e.g. under the trademark TAXOTERE. Vinblastine sulfate can be administered, e.g., in the form as it is marketed, e.g. under the trademark VINBLASTIN R.P. Vincristine sulfate can be administered, e.g., in the form as it is marketed, e.g. under the trademark FARMISTIN. Discodermolide can be obtained, e.g., as disclosed in U.S. Pat. No. 5,010,099. Also included are Epothilone derivatives which are disclosed in WO 98/10121, U.S. Pat. No. 6,194,181, WO 98/25929, WO 98/08849, WO 99/43653, WO 98/22461 and WO 00/31247. Especially preferred are Epothilone A and/or B.

The term “alkylating agent” as used herein includes, but is not limited to, cyclophosphamide, ifosfamide, melphalan or nitrosourea (BCNU or Gliadel). Cyclophosphamide can be administered, e.g., in the form as it is marketed, e.g. tinder the trademark CYCLOSTIN. Ifosfamide can be administered, e.g., in the form as it is marketed, e.g. under the trademark HOLOXAN.

The term “platin compound” as used herein includes, but is not limited to, carboplatin, cisplatin, cisplatinum and oxaliplatin. Carboplatin can be administered, e.g., in the form as it is marketed, e.g. under the trademark CARBOPLAT. Oxaliplatin can be administered, e.g., in the form as it is marketed, e.g. under the trademark ELOXATIN.

The term “compounds targeting/decreasing a protein or lipid kinase activity; or a protein or lipid phosphatase activity; or further anti-angiogenic compounds” as used herein includes, but is not limited to, protein tyrosine kinase and/or serine and/or threonine kinase inhibitors or lipid kinase inhibitors, e.g.,
a) compounds targeting, decreasing or inhibiting the activity of the platelet-derived growth factor-receptors (PDGFR), such as compounds which target, decrease or inhibit the activity of PDGFR, especially compounds which inhibit the PDGF receptor, e.g. a N-phenyl-2-pyrimidine-amine derivative, e.g. imatinib, SU101, SU6668 and GFB-111;
b) compounds targeting, decreasing or inhibiting the activity of the fibroblast growth factor-receptors (FGFR);
c) compounds targeting, decreasing or inhibiting the activity of the insulin-like growth factor receptor I (IGF-IR), such as compounds which target, decrease or inhibit the activity of IGF-IR, especially compounds which inhibit the kinase activity of IGF-I receptor, such as those compounds disclosed in WO 02/092599, or antibodies that target the extracellular domain of IGF-I receptor or its growth factors;
d) compounds targeting, decreasing or inhibiting the activity of the Trk receptor tyrosine kinase family, or ephrin B4 inhibitors;
e) compounds targeting, decreasing or inhibiting the activity of the Axl receptor tyrosine kinase family;
f) compounds targeting, decreasing or inhibiting the activity of the Ret receptor tyrosine kinase;
g) compounds targeting, decreasing or inhibiting the activity of the Kit/SCFR receptor tyrosine kinase, e.g. imatinib;
h) compounds targeting, decreasing or inhibiting the activity of the C-kit receptor tyrosine kinases—(part of the PDGFR family), such as compounds which target, decrease or inhibit the activity of the c-Kit receptor tyrosine kinase family, especially compounds which inhibit the c-Kit receptor, e.g. imatinib;
i) compounds targeting, decreasing or inhibiting the activity of members of the c-Abl family, their gene-fusion products (e.g. BCR-Abl kinase) and mutants, such as compounds which target decrease or inhibit the activity of c-Abl family members and their gene fusion products, e.g. a N-phenyl-2-pyrimidine-amine derivative, e.g. imatinib or nilotinib (AMN107); P0180970; AG957; NSC 680410; PD173955 from ParkeDavis; or dasatinib (BMS-354825);
j) compounds targeting, decreasing or inhibiting the activity of members of the protein kinase C(PKC) and Raf family of serine/threonine kinases, members of the MEK, SRC, JAK, FAK, PDK1, PKB/Akt, and Ras/MAPK family members, and/or members of the cyclin-dependent kinase family (CDK) and are especially those staurosporine derivatives disclosed in U.S. Pat. No. 5,093,330, e.g. midostaurin; examples of further compounds include e.g. UCN-01, safingol, BAY 43-9006, Bryostatin 1, Perifosine; Ilmolosine; RO 318220 and RO 320432; GO 6976; Isis 3521; LY333531/LY379196; isochinoline compounds such as those disclosed in WO 00/09495; FT's; PD184352 or QAN697 (a P13K inhibitor) or AT7519 (CDK inhibitor);
k) compounds targeting, decreasing or inhibiting the activity of protein-tyrosine kinase inhibitors, such as compounds which target, decrease or inhibit the activity of protein-tyrosine kinase inhibitors include imatinib mesylate (GLEEVEC) or tyrphostin. A tyrphostin is preferably a low molecular weight (Mr<1500) compound, or a pharmaceutically acceptable salt thereof, especially a compound selected from the benzylidenemalonitrile class or the S-arylbenzenemalonirile or bisubstrate quinoline class of compounds, more especially any compound selected from the group consisting of Tyrphostin A23/RG-50810; AG 99; Tyrphostin AG 213; Tyrphostin AG 1748; Tyrphostin AG 490; Tyrphostin B44; Tyrphostin B44 (+) enantiomer; Tyrphostin AG 555; AG 494; Tyrphostin AG 556, AG957 and adaphostin (4-{[(2,5-dihydroxyphenyl)methyl]amino}-benzoic acid adamantyl ester; NSC 680410, adaphostin);
l) compounds targeting, decreasing or inhibiting the activity of the epidermal growth factor family of receptor tyrosine kinases (EGFR, ErbB2, ErbB3, ErbB4 as homo- or heterodimers) and their mutants, such as compounds which target, decrease or inhibit the activity of the epidermal growth factor receptor family are especially compounds, proteins or antibodies which inhibit members of the EGF receptor tyrosine kinase family, e.g. EGF receptor, ErbB2. ErbB3 and ErbB4 or bind to EGF or EGF related ligands, and are in particular those compounds, proteins or monoclonal antibodies generically and specifically disclosed in WO 97/02266, e.g. the compound of ex. 39, or in EP 0 564 409, WO 99/03854, EP 0520722, EP 0 566 226, EP 0 787 722, EP 0 837 063, U.S. Pat. No. 5,747,498, WO 98/10767, WO 97/30034, WO 97/49688, WO 97/38983 and, especially, WO 96/30347 (e.g. compound known as CP 358774), WO 96/33980 (e.g. compound ZD 1839) and WO 95/03283 (e.g. compound ZM105180); e.g. trastuzumab (Herceptin™), cetuximab (Erbitux™), Iressa, Tarceva, OSI-774, Cl-1033, EKB-569, GW-2016, E1.1, E2.4, E2.5, E6.2, E6.4, E2.11, E6.3 or E7.6.3, and 7H-pyrrolo-[2,3-d]pyrimidine derivatives which are disclosed in WO 03/013541; and
m) compounds targeting, decreasing or inhibiting the activity of the c-Met receptor, such as compounds which target, decrease or inhibit the activity of c-Met, especially compounds which inhibit the kinase activity of c-Met receptor, or antibodies that target the extracellular domain of c-Met or bind to HGF.

Further anti-angiogenic compounds include compounds having another mechanism for their activity, e.g. unrelated to protein or lipid kinase inhibition e.g. thalidomide (THALOMID) and TNP-470.

Compounds which target, decrease or inhibit the activity of a protein or lipid phosphatase are e.g. inhibitors of phosphatase 1, phosphatase 2A, or CDC25, e.g. okadaic acid or a derivative thereof.

Compounds which induce cell differentiation processes are e.g. retinoic acid, α- γ- or δ-tocopherolor α- γ- or δ-tocotrienol.

The term cyclooxygenase inhibitor as used herein includes, but is not limited to, e.g. Cox-2 inhibitors, 5-alkyl substituted 2-arylaminophenylacetic acid and derivatives, such as celecoxib (CELEBREX), rofecoxib (VIOXX), etoricoxib, valdecoxib or a 5-alkyl-2-arylaminophenylacetic acid, e.g. 5-methyl-2-(2′-chloro-6′-fluoroanilino)phenyl acetic acid, lumiracoxib.

The term “bisphosphonates” as used herein includes, but is not limited to, etridonic, clodronic, tiludronic, pamidronic, alendronic, ibandronic, risedronic and zoledronic acid. “Etridonic acid” can be administered, e.g., in the form as it is marketed, e.g. under the trademark DIDRONEL. “Clodronic acid” can be administered, e.g., in the form as it is marketed, e.g. under the trademark BONEFOS. “Tiludronic acid” can be administered, e.g., in the form as it is marketed, e.g. under the trademark SKELID. “Pamidronic acid” can be administered, e.g. in the form as it is marketed, e.g. under the trademark AREDIA™. “Alendronic acid” can be administered, e.g., in the form as it is marketed, e.g. under the trademark FOSAMAX. “Ibandronic acid” can be administered, e.g., in the form as it is marketed, e.g. under the trademark BONDRANAT. “Risedronic acid” can be administered, e.g., in the form as it is marketed, e.g. under the trademark ACTONEL. “Zoledronic acid” can be administered, e.g. in the form as it is marketed, e.g. under the trademark ZOMETA, The term “mTOR inhibitors” relates to compounds which inhibit the mammalian target of rapamycin (mTOR) and which possess antiproliferative activity such as sirolimus (Rapamune™), everolimus (Certican™), CCl-779 and ABT578.

The term “heparanase inhibitor” as used herein refers to compounds which target, decrease or inhibit heparin sulfate degradation. The term includes, but is not limited to, PI-88.

The term “biological response modifier” as used herein refers to a lymphokine or interferons, e.g. interferon γ.

The term “inhibitor of Ras oncogenic isoforms”, e.g. H-Ras, K-Ras, or N-Ras, as used herein refers to compounds which target, decrease or inhibit the oncogenic activity of Ras e.g. a “farnesyl transferase inhibitor” e.g. L-744832, DK8G557 or R115777 (Zarnestra).

The term “telomerase inhibitor” as used herein refers to compounds which target, decrease or inhibit the activity of telomerase. Compounds which target, decrease or inhibit the activity of telomerase are especially compounds which inhibit the telomerase receptor. e.g. telomestatin.

The term “methionine aminopeptidase inhibitor” as used herein refers to compounds which target, decrease or inhibit the activity of methionine aminopeptidase. Compounds which target, decrease or inhibit the activity of methionine aminopeptidase are e.g. bengamide or a derivative thereof.

The term “proteasome inhibitor” as used herein refers to compounds which target, decrease or inhibit the activity of the proteasome. Compounds which target, decrease or inhibit the activity of the proteasome include e.g. Bortezomid (Velcade™) and MLN 341. The term “matrix metalloproteinase inhibitor” or (“MMP” inhibitor) as used herein includes, but is not limited to, collagen peptidomimetic and nonpeptidomimetic inhibitors, tetracycline derivatives, e.g. hydroxamate peptidomimetic inhibitor batimastat and its orally bioavailable analogue marimastat (BB-2516), prinomastat (AG3340), metastat (NSC 683551) BMS-279251, BAY 12-9566, TAA211, MMI270B or AAJ996.

The term “compounds used in the treatment of hematologic malignancies” as used herein includes, but is not limited to, FMS-like tyrosine kinase inhibitors e.g. compounds targeting, decreasing or inhibiting the activity of FMS-like tyrosine kinase receptors (Flt-3R); interferon, 1-b-D-arabinofuransylcytosine (ara-c) and bisulfan; and ALK inhibitors e.g. compounds which target, decrease or inhibit anaplastic lymphoma kinase. Compounds which target, decrease or inhibit the activity of FMS-like tyrosine kinase receptors (Flt-3R) are especially compounds, proteins or antibodies which inhibit members of the Flt-3R receptor kinase family, e.g. PKC412, midostaurin, a staurosporine derivative, SU11248 and MLN518.

The term “HSP90 inhibitors” as used herein includes, but is not limited to, compounds targeting, decreasing or inhibiting the intrinsic ATPase activity of HSP90; degrading, targeting, decreasing or inhibiting the HSP90 client proteins via the ubiquitin proteosome pathway. Compounds targeting, decreasing or inhibiting the intrinsic ATPase activity of HSP90 are especially compounds, proteins or antibodies which inhibit the ATPase activity of HSP90 e.g., 17-allylamino,17-demethoxygeldanamycin (17AAG), a geldanamycin derivative; other geldanamycin related compounds; radicicol and HDAC inhibitors.

The term “antiproliferative antibodies” as used herein includes, but is not limited to, trastuzumab (Herceptin™), Trastuzumab-DM1, erbitux, bevacizumab (Avastin™), rituximab (Rituxan®), PRO64553 (anti-CD40) and 2C4 Antibody. By antibodies is meant e.g. intact monoclonal antibodies, polyclonal antibodies, multispecific antibodies formed from at least 2 intact antibodies, and antibodies fragments so long as they exhibit the desired biological activity.

The term “antileukemic compounds” includes, for example, Ara-C, a pyrimidine analog, which is the 2′-alpha-hydroxy ribose (arabinoside) derivative of deoxycytidine, Also included is the purine analog of hypoxanthine, 6-mercaptopurine (6-MP) and fludarabine phosphate. Somatostatin receptor antagonists as used herein refers to compounds which target, treat or inhibit the somatostatin receptor such as octreotide, and SOM230.

Tumor cell damaging approaches refer to approaches such as ionizing radiation. The term “ionizing radiation” referred to above and hereinafter means ionizing radiation that occurs as either electromagnetic rays (such as X-rays and gamma rays) or particles (such as alpha and beta particles). Ionizing radiation is provided in, but not limited to, radiation therapy and is known in the art. See Hellman, Principles of Radiation Therapy, Cancer, in Principles and Practice of Oncology, Devita et al., Eds., 4^{th} Edition, Vol. 1, pp. 248-275 (1993).

The term EDG binders as used herein refers a class of immunosuppressants that modulates lymphocyte recirculation, such as FTY720.

The term ribonucleotide reductase inhibitors refers to pyrimidine or purine nucleoside analogs including, but not limited to, fludarabine and/or cytosine arabinoside (ara-C), 6-thioguanine, 5-fluorouracil, cladribine, 6-mercaptopurine (especially in combination with ara-C against ALL) and/or pentostatin. Ribonucleotide reductase inhibitors are especially hydroxyurea or 2-hydroxy-1H-isoindole-1,3-dione derivatives, such as PL-1, PL-2, PL-3, PL-4, PL-5, PL-6, PL-7 or PL-8 mentioned in Nandy et al., Acta Oncologica, Vol. 33, No. 8, pp. 953-961 (1994).

The term “S-adenosylmethionine decarboxylase inhibitors” as used herein includes, but is not limited to the compounds disclosed in U.S. Pat. No. 5,461,076.

Also included are in particular those compounds, proteins or monoclonal antibodies of VEGF disclosed in WO 98/35958, e.g. 1-(4-chloroanilino)-4-(4-pyridylmethyl)phthalazine or a pharmaceutically acceptable salt thereof, e.g. the succinate, or in WO 00/09495, WO 00/27820, WO 00/59509, WO 98/11223, WO 00/27819 and EP 0 769 947; those as described by Prewett et al, Cancer Res, Vol. 59, pp. 5209-5218 (1999); Yuan et al., Proc Natl Acad Sci USA, Vol. 93, pp. 14765-14770 (1996); Zhu et al., Cancer Res, Vol. 58, pp, 3209-3214 (1998); and Mordenti et al., Toxicol Pathol, Vol. 27, No. 1, pp. 14-21 (1999); in WO 00/37502 and WO 94/10202; ANGIOSTATIN, described by O'Reilly et al., Cell, Vol. 79, pp. 315-328 (1994); ENDOSTATIN, described by O'Reilly et al., Cell, Vol. 88, pp. 277-285 (1997); anthranilic acid amides; ZD4190; ZD6474; SU5416; SU6668; bevacizumab; or anti-VEGF antibodies or anti-VEGF receptor antibodies, e.g. rhuMAb and RHUFab, VEGF aptamer e.g. Macugon; FLT-4 inhibitors, FLT-3 inhibitors, VEGFR-2 IgG1 antibody, Angiozyme (RPI 4610) and Bevacizumab (Avastin™).

Photodynamic therapy as used herein refers to therapy which uses certain chemicals known as photosensitizing compounds to treat or prevent cancers. Examples of photodynamic therapy includes treatment with compounds, such as e.g. VISUDYNE and porfimer sodium.

Angiostatic steroids as used herein refers to compounds which block or inhibit angiogenesis, such as, e.g., anecortave, triamcinolone, hydrocortisone, 11-α-epihydrocotisol, cortexolone, 17α-hydroxyprogesterone, corticosterone, desoxycorticosterone, testosterone, estrone and dexamethasone.

Implants containing corticosteroids refers to compounds, such as e.g. fluocinolone, dexamethasone.

Other chemotherapeutic compounds include, but are not limited to, plant alkaloids, hormonal compounds and antagonists; biological response modifiers, preferably lymphokines or interferons; antisense oligonucleotides or oligonucleotide derivatives; shRNA or siRNA; or miscellaneous compounds or compounds with other or unknown mechanism of action.

The compounds of the invention are also useful as co-therapeutic compounds for use in combination with other drug substances such as anti-inflammatory, bronchodilatory or antihistamine drug substances, particularly in the treatment of obstructive or inflammatory airways diseases such as those mentioned hereinbefore, for example as potentiators of therapeutic activity of such drugs or as a means of reducing required dosaging or potential side effects of such drugs. A compound of the invention may be mixed with the other drug substance in a fixed pharmaceutical composition or it may be administered separately, before, simultaneously with or after the other drug substance. Accordingly the invention includes a combination of a compound of the invention as hereinbefore described with an anti-inflammatory, bronchodilatory, antihistamine or anti-tussive drug substance, said compound of the invention and said drug substance being in the same or different pharmaceutical composition.

Suitable anti-inflammatory drugs include steroids, in particular glucocorticosteroids such as budesonide, beclamethasone dipropionate, fluticasone propionate, ciclesonide or mometasone furoate, or steroids described in WO 02/88167, WO 02/12266, WO 02/100879, WO 02/00679 (especially those of Examples 3, 11, 14, 17, 19, 26, 34, 37, 39, 51, 60, 67, 72, 73, 90, 99 and 101), WO 03/035668, WO 03/048181, WO 03/062259, WO 03/064445, WO 03/072592, non-steroidal glucocorticoid receptor agonists such as those described in WO 00/00531, WO 02/10143, WO 03/082280, WO 03/082787, WO 03/104195, WO 04/005229; LTB4 antagonists such LY293111, CGS025019C, CP-195543, SC-53228, BIIL 284, ONO 4057, SB 209247 and those described in U.S. Pat. No. 5,451,700; LTD4 antagonists such as montelukast and zafirlukast; PDE4 inhibitors such cilomilast (Ariflo® GlaxoSmithKline), Roflumilast (Byk Gulden), V-11294A (Napp), BAY19-8004 (Bayer), SCH-351591 (Schering-Plough), Arofylline (Almirall Prodesfarma), PD189659/PD168787 (Parke-Davis), AWD-12-281 (Asta Medica), CDC-801 (Celgene), SelCID™ CC-10004 (Celgene), VM554/UM565 (Vernalis), T-440 (Tanabe), KW-4490 (Kyowa Hakko Kogyo), and those disclosed in WO 92/19594, WO 93/19749, WO 93/19750, WO 93/19751, WO 98/18796, WO 99/16766, WO 01/13953, WO 03/104204, WO 03/104205, WO 03/39544, WO 04/000814, WO 04/000839, WO 04/005258, WO 04/018450, WO 04/018451, WO 04/018457, WO 04/018465, WO 04/018431, WO 04/018449, WO 04/018450, WO 04/018451, WO 04/018457, WO 04/018465, WO 04/019944, WO 04/019945, WO 04/045607 and WO 04/037805; A2a agonists such as those disclosed in EP 409595A2, EP 1052264, EP 1241176, WO 94/17090, WO 96/02543, WO 96/02553, WO 98/28319, WO 99/24449, WO 99/24450, WO 99/24451, WO 99/38877, WO 99/41267, WO 99/67263, WO 99/67264, WO 99/67265, WO 99/67266, WO 00/23457, WO 00/77018, WO 00/78774, WO 01/23399, WO 01/27130, WO 01/27131, WO 01/60835, WO 01/94368, WO 02/00676, WO 02/22630, WO 02/96462, WO 03/086408, WO 04/039762, WO 04/039766, WO 04/045618 and WO 04/046083; A2b antagonists such as those described in WO 02/42298; and beta-2 adrenoceptor agonists such as albuterol (salbutamol), metaproterenol, terbutaline, salmeterol fenoterol, procaterol, and especially, formoterol and pharmaceutically acceptable salts thereof, and compounds (in free or salt or solvate form) of formula I of WO 0075114, which document is incorporated herein by reference, preferably compounds of the Examples thereof, especially a compound of formula

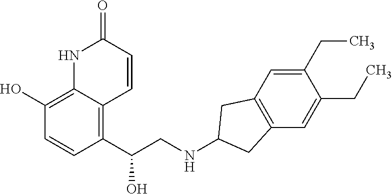

and pharmaceutically acceptable salts thereof, as well as compounds (in free or salt or solvate form) of formula I of WO 04/16601, and also compounds of WO 04/033412. Suitable bronchodilatory drugs include anticholinergic or antimuscarinic compounds, in particular ipratropium bromide, oxitropium bromide, tiotropium salts and CHF 4226 (Chiesi), and glycopyrrolate, but also those described in WO 01/04118, WO 02/51841, WO 02/53564, WO 03/00840, WO 03/87094, WO 04/05285, WO 02/00652, WO 03/53966, EP 424021, U.S. Pat. No. 5,171,744, U.S. Pat. No. 3,714,357, WO 03/33495 and WO 04/018422.

Suitable antihistamine drug substances include cetirizine hydrochloride, acetaminophen, clemastine fumarate, promethazine, loratidine, desloratidine, diphenhydramine and fexofenadine hydrochloride, activastine, astemizole, azelastine, ebastine, epinasfine, mizolastine and tefenadine as well as those disclosed in WO 03/099807, WO 04/026841 and JP 2004107299.

Other useful combinations of compounds of the invention with anti-inflammatory drugs are those with antagonists of chemokine receptors, e.g. CCR-1, CCR-2, CCR-3, CCR-4, CCR-5, CCR-6, CCR-7, CCR-8, CCR-9 and CCR10, CXCR1, CXCR2, CXCR3, CXCR4, CXCR5, particularly CCR-5 antagonists such as Schering-Plough antagonists SC-351125, SCH-55700 and SCH-D, Takeda antagonists such as N-[[4-[[[6,7-dihydro-2-(4-methylphenyl)-5H-benzo-cyclohepten-8-yl]carbonyl]amino]phenyl]-methyl]tetrahydro-N,N-dimethyl-2H-pyran-4-amin-ium chloride (TAK-770), and CCR-5 antagonists described in U.S. Pat. No. 6,166,037 (particularly claims 18 and 19), WO 00/66558 (particularly claim 8), WO 00/66559 (particularly claim 9), WO 04/018425 and WO 04/026873.

The structure of the active compounds identified by code nos., generic or trade names may be taken from the actual edition of the standard compendium The Merck Index or from databases, e.g. Patents International (e.g. IMS World Publications).

The above-mentioned compounds, which can be used in combination with a compound of the formula (I), can be prepared and administered as described in the art, such as in the documents cited above.

A compound of the formula (I) may also be used to advantage in combination with known therapeutic processes, for example, the administration of hormones or especially radiation.

A compound of formula (I) may in particular be used as a radiosensitizer, especially for the treatment of tumors which exhibit poor sensitivity to radiotherapy.

By “combination”, there is meant either a fixed combination in one dosage unit form, or a kit of parts for the combined administration where a compound of the formula (I) and a combination partner may be administered independently at the same time or separately within time intervals that especially allow that the combination partners show a cooperative, e.g. synergistic effect.

### EXAMPLES

The preclinical activity of LBH589 tested against proliferation and viability of 3 Hodgkin lymphoma cell lines, RPMI6666, HD-MY-Z, and L428.
HL cell lines exhibited extreme sensitivity to LBH589 with IC₅₀ values ranging from 200 picomolar to 50 nanomolar
Cell lines of mantle cell and follicular lymphoma demonstrate extreme sensitivity to LBh589
These concentrations can easily be achieved with doses that can be safely administered in human studies
CTCL cell lines are extremely sensitive to LBH589. The pan-DAC inhibitor produces profound tumor regression in a CTCL mouse model.
LBH589 produces regression of the E□-myc mouse lymphoma model and extends survival.
B-cell lymphoma protein-2 (Bcl2) mediates resistance to LBH589 in E□-myc mouse tumors. Bcl2 may be a patient stratification marker

Single agent anti-tumor activity of LBH589 in Hodgkins Lymphoma cell lines in vitro is outlined in table I.

TABLE I

| | Disease | | | |
|---|---|---|---|---|
| Cell line | type | IC50 | LD50 | LD90 |
| RPMI 6666 | Hodgkins | 0.2 | 6.7 | 274.7 |
| | Lymphoma | | | |
| HD-MY-Z | Hodgkins | 50.8 | 239.5 | >10,000 |
| | Lymphoma | | | |
| L-428 | Hodgkins | 10.5 | 26.1 | 57.5 |
| | Lymphoma | | | |

IC50: Concentration of LBH589 required to inhibit growth of cell line by 50%
LD50: Concentration of LBH589 required to kill a cell line population by 50%
LD90: Concentration of LBH589 required to kill a cell line population by 90%

1-6. (canceled)

7. A method of treating Hodgkins disease comprising administering a therapeutically effective amount of an HDAC inhibitor to a warm-blooded animal in need thereof.

8. A method according to claim 7, comprising administering a therapeutically effective amount of a compound of formula (I): 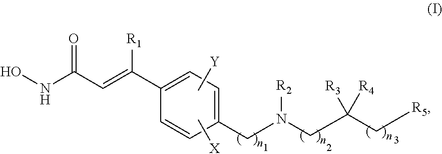
wherein
R₁ is H; halo; or a straight-chain C₁-C₆alkyl, especially methyl, ethyl or n-propyl, which methyl, ethyl and n-propyl substituents are unsubstituted or substituted by one or more substituents described below for alkyl substituents;
R₂ is selected from H; C₁-C₁₀alkyl, preferably C₁-C₆alkyl, e.g., methyl, ethyl or —CH₂CH₂—OH; C₄-C₉cycloalkyl; C₄-C₉heterocycloalkyl; C₄-C₉heterocycloalkylalkyl; cycloalkylalkyl, e.g., cyclopropylmethyl; aryl; heteroaryl; arylalkyl, e.g., benzyl; heteroarylalkyl, e.g., pyridylmethyl; —(CH₂)ₙC(O)R₆; —(CH₂)ₙOC(O)R₆, amino acyl; HON—C(O)—CH═C(R₁)-aryl-alkyl-; and —(CH₂)ₙR₇;
R₃ and R₄ are the same or different and, independently, H; C₁-C₆alkyl; acyl; or acylamino; or
R₃ and R₄, together with the carbon to which they are bound, represent C═O, C═S or C═NR₈; or
R₂, together with the nitrogen to which it is bound, and R₃, together with the carbon to which it is bound, can form a C₄-C₉heterocycloalkyl; a heteroaryl; a polyheteroaryl; a non-aromatic polyheterocycle; or a mixed aryl and non-aryl polyheterocycle ring;
R₅ is selected from H; C₁-C₆alkyl; C₄-C₉cycloalkyl; C₄-C₉heterocycloalkyl; acyl; aryl; heteroaryl; arylalkyl, e.g., benzyl; heteroarylalkyl, e.g., pyridylmethyl; aromatic polycycles; non-aromatic polycycles; mixed aryl and non-aryl polycycles; polyheteroaryl; non-aromatic polyheterocycles; and mixed aryl and non-aryl polyheterocycles;
n, n₁, n₂ and n₃ are the same or different and independently selected from 0-6, when n₁ is 1-6, each carbon atom can be optionally and independently substituted with R₃ and/or R₄;
X and Y are the same or different and independently selected from H; halo; C₁-C₄alkyl, such as CH₃ and CF₃; NO₂; C(O)R₁; OR₉; SR₉; CN; and NR₁₀R₁₁;
R₆ is selected from H; C₁-C₆alkyl; C₄-C₉cycloalkyl; C₄-C₉heterocycloalkyl; cycloalkylalkyl, e.g., cyclopropylmethyl; aryl; heteroaryl; arylalkyl, e.g., benzyl and 2-phenylethenyl; heteroarylalkyl, e.g., pyridylmethyl; OR₁₂; and NR₁₃R₁₄;
R₇ is selected from OR₁₅; SR₁₅; S(O)R₁₆; SO₂R₁₇; NR₁₃R₁₄; and NR₁₂SO₂R₆;
R₈ is selected from H; OR₁₅; NR₁₃R₁₄; C₁-C₆alkyl; C₄-C₉cycloalkyl; C₄-C₉heterocycloalkyl; aryl; heteroaryl; arylalkyl, e.g., benzyl; and heteroarylalkyl, e.g., pyridylmethyl;
R₉ is selected from C₁-C₄alkyl, e.g., CH₃ and CF₃; C(O)-alkyl, e.g., C(O)CH₃; and C(O)CF₃;
R₁₀ and R₁₁ are the same or different and independently selected from H; C₁-C₄alkyl; and —C(O)-alkyl;
R₁₂ is selected from H; C₁-C₆alkyl; C₄-C₉cycloalkyl; C₄-C₉heterocycloalkyl; C₄-C₉heterocycloalkylalkyl; aryl; mixed aryl and non-aryl polycycle; heteroaryl; arylalkyl, e.g., benzyl; and heteroarylalkyl, e.g., pyridylmethyl;
R₁₃ and R₁₄ are the same or different and independently selected from H; C₁-C₆alkyl; C₄-C₉cycloalkyl; C₄-C₉heterocycloalkyl; aryl; heteroaryl; arylalkyl, e.g., benzyl; heteroarylalkyl, e.g., pyridylmethyl; amino acyl; or
R₁₃ and R₁₄, together with the nitrogen to which they are bound, are C₄-C₉heterocycloalkyl; heteroaryl; polyheteroaryl; non-aromatic polyheterocycle; or mixed aryl and non-aryl polyheterocycle;
R₁₅ is selected from H; C₁-C₆alkyl; C₄-C₉cycloalkyl; C₄-C₉heterocycloalkyl; aryl; heteroaryl; arylalkyl; heteroarylalkyl; and (CH₂)ₘZR₁₂;
R₁₆ is selected from C₁-C₆alkyl; C₄-C₉cycloalkyl; C₄-C₉heterocycloalkyl; aryl; heteroaryl; polyheteroaryl; arylalkyl; heteroarylalkyl; and (CH₂)ₘZR₁₂;
R₁₇ is selected from C₁-C₆alkyl; C₄-C₉cycloalkyl; C₄-C₉heterocycloalkyl; aryl; aromatic polycycles; heteroaryl; arylalkyl; heteroarylalkyl; polyheteroaryl and NR₁₃R₁₄;
m is an integer selected from 0-6; and
Z is selected from 0; NR₁₃; S; and S(O),
or a pharmaceutically acceptable salt thereof to a warm-blooded animal in need thereof.

9. A method according to claim 7, wherein the compound of formula (I) is N-hydroxy-3-[4-[[[2-(2-methyl-1H-indol-3-yl)-ethyl]amino]methyl]phenyl]-2E-2-propenamide haying the formula (III): 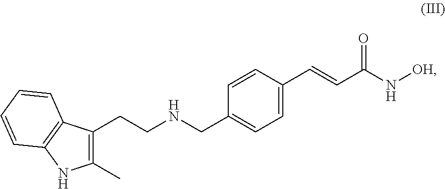
or a pharmaceutically acceptable salt thereof.

10. A method according to claim 7, wherein the Hodgkins disease is a lymphoproliferative disease characterized by characterized clinically by the orderly spread of disease from one lymph node group to another and by the development of systemic symptoms with advanced disease.

11. A method according to claim 7, wherein the warm-blooded animal is a human.

12. A method according to claim 7, wherein the disease is Hodgkins disease.
